(19) United States
(12) Patent Application Publication
(10) Pub. No.: US 2022/0202484 A1
(43) Pub. Date: Jun. 30, 2022

(54) RF ABLATION SYSTEMS AND METHODS USING A REMOTE OR IN-LINE CONTROLLER

(71) Applicant: Boston Scientific Neuromodulation Corporation, Valencia, CA (US)

(72) Inventors: Kevin Peng Wang, Fremont, CA (US); Darragh McDermott, Los Angeles, CA (US)

(21) Appl. No.: 17/553,527

(22) Filed: Dec. 16, 2021

Related U.S. Application Data
(60) Provisional application No. 63/130,519, filed on Dec. 24, 2020.

(51) Int. Cl. A61B 18/14; A61B 18/12; G16H 40/67

(52) U.S. Cl. CPC A61B 18/1492; A61B 2018/00577; G16H 40/67; A61B 18/1206

(57) ABSTRACT

A RF ablation system includes a RF electrode coupleable to a RF generator for delivering RF energy from the RF generator to patient tissue for ablation, the RF electrode including an electrode hub, an electrode shaft extending from the electrode hub, and a cable extending from the electrode hub. The RF ablation system also includes an in-line controller coupleable to the RF generator for controlling the delivering of the RF energy from the RF generator along the RF electrode, wherein the in-line controller is configured for placement nearer the electrode hub than the RF generator and includes at least one actuator for controlling the delivering of the RF energy.

### CROSS-REFERENCE TO RELATED APPLICATIONS

This application claims the benefit under 35 U.S.C. § 119(e) of U.S. Provisional Patent Application Ser. No. 63/130,519, filed Dec. 24, 2020, which is incorporated herein by reference.

### FIELD

The present disclosure is directed to the area of radiofrequency (RF) ablation systems and methods of making and using the systems. The present disclosure is also directed to RF ablation system and methods that include a remote or in-line controller, as well as methods of making and using the same.

### BACKGROUND

Radiofrequency (RF) generators and electrodes can be used for pain relief or functional modification. Radiofrequency ablation (RFA) is a safe, proven means of interrupting pain signals, such as those coming from irritated facet joints in the spine, genicular nerves in the knee, and femoral and obturator nerves in the hip. Radiofrequency current is used to heat up a small volume of nerve tissue, thereby interrupting pain signals from that specific area. Radiofrequency ablation is designed to provide long-lasting pain relief.

For example, an RF electrode can be positioned near target tissue and then used to heat the target tissue by RF power dissipation of the RF signal output in the target tissue. Temperature monitoring of the target tissue by a temperature sensor in the electrode may be used to control the process.

### BRIEF SUMMARY

One aspect is a RF ablation system that includes a RF electrode coupleable to a RF generator for delivering RF energy from the RF generator to patient tissue for ablation, the RF electrode including an electrode hub, an electrode shaft extending from the electrode hub, and a cable extending from the electrode hub. The RF ablation system also includes an in-line controller coupleable to the RF generator for controlling the delivering of the RF energy from the RF generator along the RF electrode, wherein the in-line controller is configured for placement nearer the electrode hub than the RF generator and includes at least one actuator for controlling the delivering of the RF energy.

In at least some aspects, the in-line controller is disposed along the cable of the RF electrode. In at least some aspects, the in-line controller is disposed in a range of 4 to 10 cm along the cable from the electrode hub.

In at least some aspects, the RF ablation system further includes an extension cable configured to couple the cable of the RF electrode to the RF generator. In at least some aspects, the in-line controller is disposed along the extension cable.

In at least some aspects, the in-line controller is disposed on the electrode hub. In at least some aspects, the at least one actuator includes a plurality of user-operable buttons. In at least some aspects, the user-operable buttons are color-coded. In at least some aspects, the user-operable buttons are squeeze sections of the cable of the RF electrode.

In at least some aspects, the in-line controller includes a controller body, wherein the at least one actuator includes a plurality of user-operable buttons disposed along the controller body. In at least some aspects, the RF electrode further includes a connector disposed at an end of the cable and the controller body includes a plurality of ports configured to receive the connector from the RF electrode. In at least some aspects, multiple ones of the user-operable buttons are individually associated with each of the ports.

In at least some aspects, the RF ablation system further includes the RF generator coupleable to the RF electrode and the in-line controller.

Another aspect is a RF electrode coupleable to a RF generator for delivering RF energy from the RF generator to patient tissue for ablation. The RF electrode includes an electrode hub; an electrode shaft extending from the electrode hub; a cable extending from the electrode hub; and an in-line controller disposed along the cable and coupleable to the RF generator for controlling the delivering of the RF energy from the RF generator along the RF electrode.

In at least some aspects, the in-line controller is disposed in a range of 4 to 10 cm along the cable from the electrode hub. In at least some aspects, the in-line controller is disposed on the electrode hub.

In at least some aspects, the at least one actuator includes a plurality of user-operable buttons. In at least some aspects, the user-operable buttons are color-coded. In at least some aspects, the user-operable buttons are squeeze sections of the cable of the RF electrode.

Yet another aspect is a remote controller for a RF generator for providing RF ablation to a patient. The remote controller includes a controller body; a plurality of user-operable buttons disposed along the controller body, wherein each of the user-operable buttons is associated with one of a plurality of RF channels of the RF generator; and a wireless communication module configured for wireless communication with the RF generator.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic side view of components of one embodiment of a RF ablation system;

FIG. 2 is a schematic side view of one embodiment of a RF electrode with an in-line controller;

FIG. 3 is a schematic view of one embodiment of an in-line controller disposed along a cable of a RF electrode;

FIG. 4 is a schematic of one embodiment of an in-line controller;

FIG. 5 is a schematic view of one embodiment of an in-line controller disposed along an extension;

FIG. 6 is a flowchart of one embodiment of a method of using an in-line controller;

FIG. 7 is a schematic perspective view of another embodiment of an in-line controller; and

FIG. 8 is a schematic perspective view of another embodiment of a remote controller using wireless communication with the RF generator.

### DETAILED DESCRIPTION

The present disclosure is directed to the area of radiofrequency (RF) ablation systems and methods of making and using the systems. The present disclosure is also directed to RF ablation system and methods that include a remote or in-line controller, as well as methods of making and using the same.

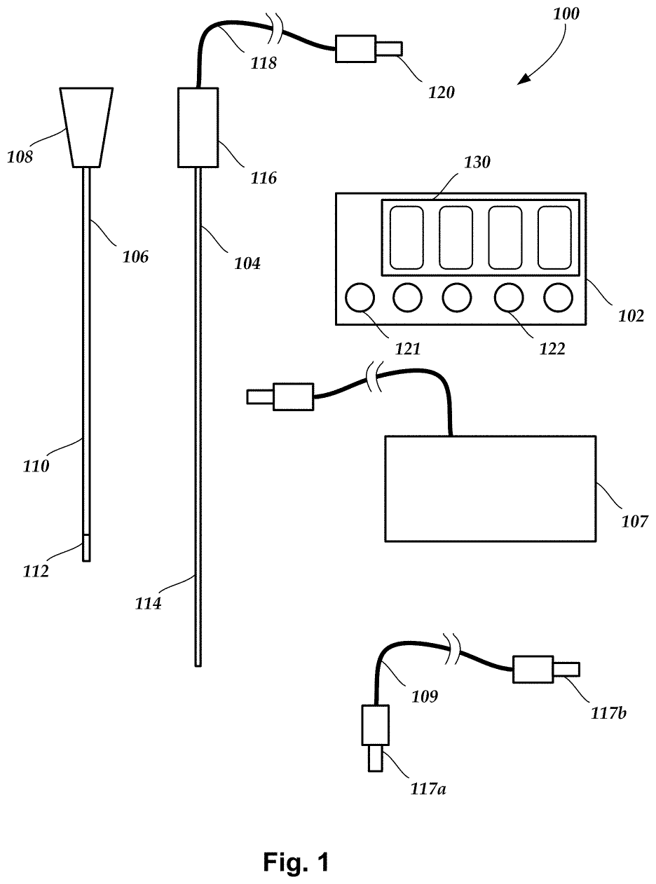

FIG. 1 illustrates one embodiment of an RF ablation system 100 that includes a RF generator 102, a RF electrode 104, a cannula 106, a ground pad 107, and an optional extension cable 109. The cannula 106 includes a cannula hub 108, an insulated shaft 110, and an active tip 112. The insulated shaft 110 is hollow for receiving the RF electrode 104. When inserted, the RF electrode 104 contacts, and energizes, the active tip 112 of the cannula 106 to produce RF ablation. The RF electrode 104 includes an electrode shaft 114, an electrode hub 116, a cable 118 that is electrically coupled to the electrode shaft 114, and a connector 120 for connecting to a port 122 of the RF generator 102 to energize the electrode shaft 114 via the cable 118 and connector 120. The optional adapter or extension 109 includes a cable 119 and connectors 117a, 117b for coupling the RF electrode 104 to the RF generator 102. It will be recognized that other RF ablation systems utilize the RF electrode 104 for ablation instead of, or in addition to, the cannula 106.

The RF generator 102 can include one or more ports 122 and at least one screen 130. In at least some embodiments, each port 122 is associated with a portion of the screen 130 (or a different screen) and can receive the connector 120 from an RF electrode 104. Information such as current, voltage, status, or the like or any combination thereof can be displayed on the screen 130. In at least some embodiments, each port 122 corresponds to an independent channel for operating a RF electrode 104. The RF generator 102 also includes a ground port 121 for attachment of the ground pad 107.

Examples of RF generators and RF ablation systems and methods of making and using the RF generators and RF ablation systems can be found at, for example, U.S. Pat. Nos. 9,717,552; 9,956,032; 10,111,703; 10,136,937; 10,136,942; 10,136,943; 10,194,971; 10,342,606; 10,363,063; 10,588,687; 10,631,915; 10,639,098; and 10,639,101 and U.S. Patent Application Publications Nos. 2014/0066917; 2014/081260; and 2014/0121658, all of which are incorporated herein by reference in their entireties.

In many RF ablation systems, there are multiple RF electrodes 104 and cannulas 106 with each RF electrode having its own cable 118 that is inserted into a different port 122 of the RF generator 102. In at least some instances, the cables 118 (with or without extensions 109) are at least two or three meters in length to provide sufficient spacing between the RF generator 102 and the patient and to avoid tension or pulling on the cable and the RF electrode 104 which could dislodge or alter the position of the RF electrode or cannula 106. Often treatment of a patient involves multiple RF electrodes 104 and cable management can be a challenge for an RF ablation system. For example, it may be challenging to identify which RF electrode 104 is attached to a particular port 122 of the RF generator 102 due, at least in part, to the length of the cables 118 (with or without extensions 109).

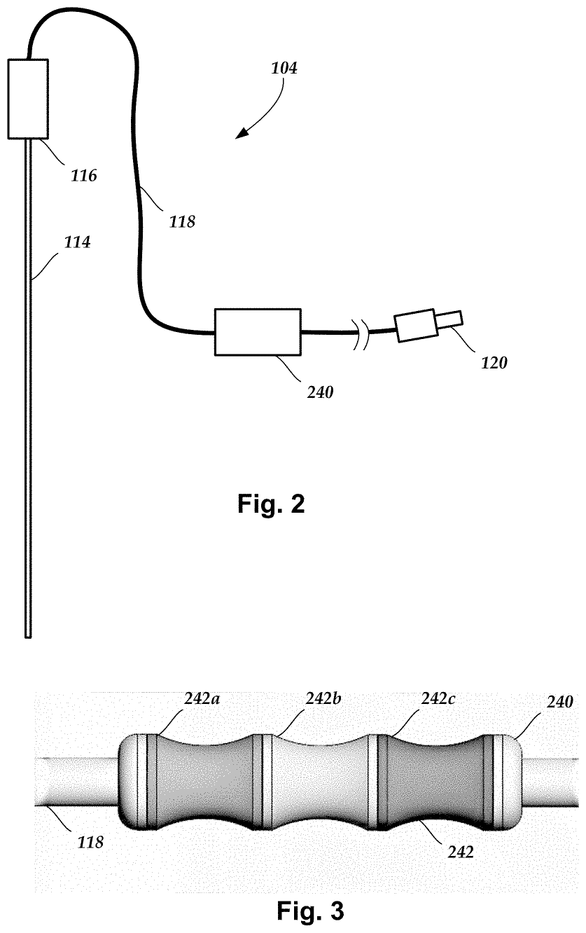

As described herein and illustrated in FIG. 2, an in-line controller 240 can be included along the cable 118 of the RF electrode 104 (or along the extension 109 as described below). The in-line controller 240 can include one or more buttons 242 (FIG. 3) or other actuators. A clinician or an assistant can operate one of the buttons 242 or other actuators to energize the electrode shaft 114 of the RF electrode 104 or to perform other functions. The in-line controller 240 can be positioned nearer to the electrode hub 116 of the RF electrode 104 for ease of identification of which RF electrode corresponds to the in-line controller. The inclusion of an in-line controller 240 may also reduce the need for an additional staff member stationed at the RF generator 102 to activate generator functions.

FIG. 3 illustrates one embodiment of an in-line controller 240 that includes multiple buttons 242a, 242b, 242c to perform multiple tasks. In at least some embodiments, the in-line controller 240 and the buttons 242a, 242b, 242c are autoclavable for reuse. In at least some embodiments, the in-line controller 240 and the buttons 242a, 242b, 242c are configured to be cleanable using liquid disinfectant.

In at least some embodiments, the buttons 242a, 242b, 242c may be color-coordinated with a function. In at least some embodiments, the color-coordinated function may also be shown on the screen 130 of the RF generator 102. For example, one button 242b may be yellow for pulsed ablation which may correspond to a yellow color on the screen 130 of the RF generator 102 for pulsed operation of the RF generator. Another button 242c may be red for thermal ablation which may correspond to a red color on the screen 130 of the RF generator 102 for thermal rf ablation using the RF generator. Another button 242a may be green which may correspond to a green color on the screen 130 of the RF generator 102 for motor stimulation testing by the RF generator. In at least some embodiments, the function(s) associated with each of the buttons 241a, 242b, 242c can be user-modifiable or user-mappable.

In at least some embodiments, a first press of a button 242 may be used select a specific function. A second press of the same button 242 may be used to indicate the intention to start the function (stimulation, ablation, pulsed operation, or the like.) A third press of the same button 242 may be used to indicate for confirmation and to start the function. For example, in at least some embodiments, a menu may pop up on the screen 130 of the RF generator 102 asking to confirm the operation at which point the user may choose to press any other button to exit or press the same button once again to confirm and begin the desired function.

The illustrated embodiment of FIG. 3 includes three buttons 242. Other embodiments can include more buttons for more functions, fewer buttons for fewer functions, one or more buttons that can be pressed different numbers of times for different functions, or the like or any combination thereof. In at least some embodiments, the buttons 242 can be colored or non-colored. In at least some embodiments, the buttons 242 can have different shapes to distinguish the buttons from each other. In at least some embodiments, the buttons 242 can be covered with an overmold of silicone or other material. In other embodiments, the buttons 242 are not covered.

In at least some embodiments, a shape of the in-line controller 240 is relatively streamlined. In at least some embodiments, a size of the in-line controller 240 is relatively small. This may limit bulkiness of the RF electrode 104. In at least some embodiments, a weight of the in-line controller 240 is selected to limit weight on the cable 118 and RF electrode 104. In at least some embodiments, the in-line controller is covered in a silicone shell to match the feel of the cable in a silicone jacket.

In at least some embodiments, the position of the in-line controller 240 is such that it is far enough from the electrode hub 116 so that the in-line controller doesn't pull on the cannula 106 or RF electrode 104 and affect the position of the cannula or the RF electrode. In addition, the in-line controller 240 is preferably sufficiently close to the electrode hub 116 to easily identify which RF electrode 104 is associated with the in-line controller. In at least some embodiments, the distance from the electrode hub 116 to the in-line controller 240 is in a range of 4 to 10 cm. In at least some embodiments, a suitable distance is selected so that the in-line controller 240 can rest on the patient and also maintain clarity as to the identity of the associated RF electrode 104.

In at least some embodiments, the buttons 242 appear to be squeeze sections of the cable 118 that trigger the desired function. In at least some embodiments, the overmold or casing of the cable 118 (such as a silicone overmold or casing) covers these buttons/squeeze sections 242. In at least some embodiments, the overmold or casing can be dyed (or otherwise colored) with certain colors to indicate the different buttons/squeeze sections 242 and possibly indicate the different functions.

In other embodiments, the buttons 242 can be located on the electrode hub 116 of the RF electrode 104.

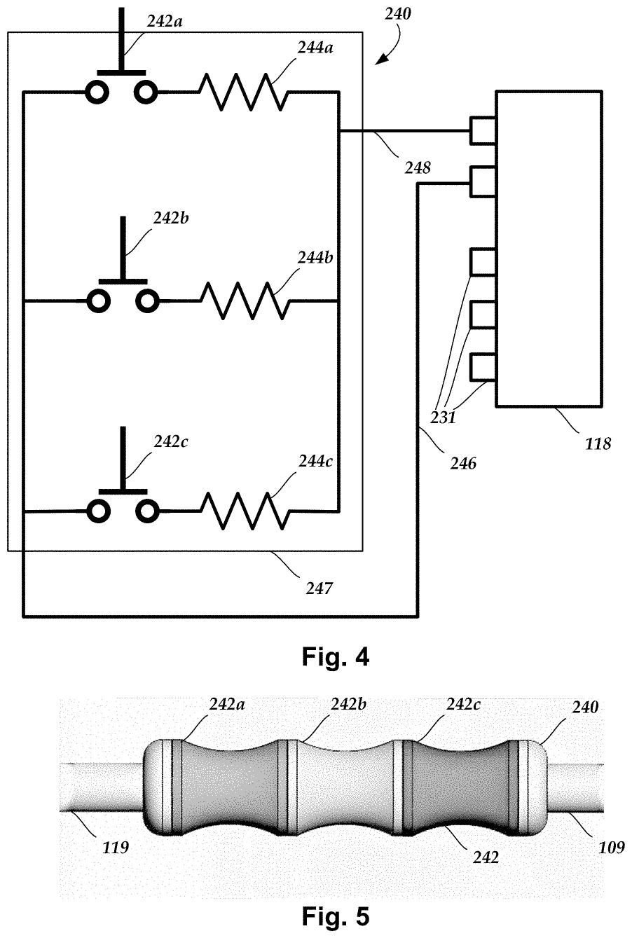

FIG. 4 is a schematic illustrating one example of the in-line controller 240 with buttons 242a, 242b, 242c that are coupled in parallel to a printed circuit board 247. Each button 242a, 242b, 242c is coupled to a different resistor 244a, 244b, 244c with a known resistance. The RF generator 102 (or other device) applies, through the cable 118, a voltage to a signal line 246 of the in-line controller 240. When a button 242a, 242b, 242c is depressed, the button shorts the signal line 246 to the return line 248 which is sensed by the RF generator 102 (or other device). The resistor 244a, 244b 244c corresponding to the depressed button 242a, 242b, 242c changes the voltage applied which identifies which button was pressed. In addition to the signal line 246 and return line 248, the cable 118 includes one or more lines 231 to the electrode, thermocouple, or other elements of the RF electrode 104. Other in-line controller arrangements can be used.

In other embodiments, the in-line controller 240 has separate contacts on the connector 120 of the RF electrode 104 and these contacts couple to circuitry in the RF generator 102 that monitors the in-line controller 240 and presses of buttons 240.

In yet other embodiments, the signal line 246 of the in-line controller 240 can send out a high frequency signal that does not affect the operation of the RF electrode 104. When the buttons 242 are depressed, it shorts the signal line to another line on the RF electrode 104. For example, the RF electrode 104 may include a RF line, a line to a thermocouple A for temperature measurement, and a line to thermocouple B for temperature measurement at a different point along the RF electrode. In one embodiment, the signal line can be short to 1) the thermocouple A line using button 242a, 2) to the thermocouple B line using button 242b, or 3) to both the thermocouple A and B lines using button 242c. The RF generator 102 can monitor the thermocouple A and B lines to determine the presses of the buttons 242a, 242b, 242c of the in-line controller 240.

Any other suitable circuitry can be used for the in-line controller 240, RF generator 102, and RF electrode 104.

In some embodiments, instead of placing the in-line controller 240 along the cable of the RF electrode 102, the in-line controller 240 can be positioned along the cable 119 (or on one of the connectors 117) of an extension 109, as illustrated in FIG. 5.

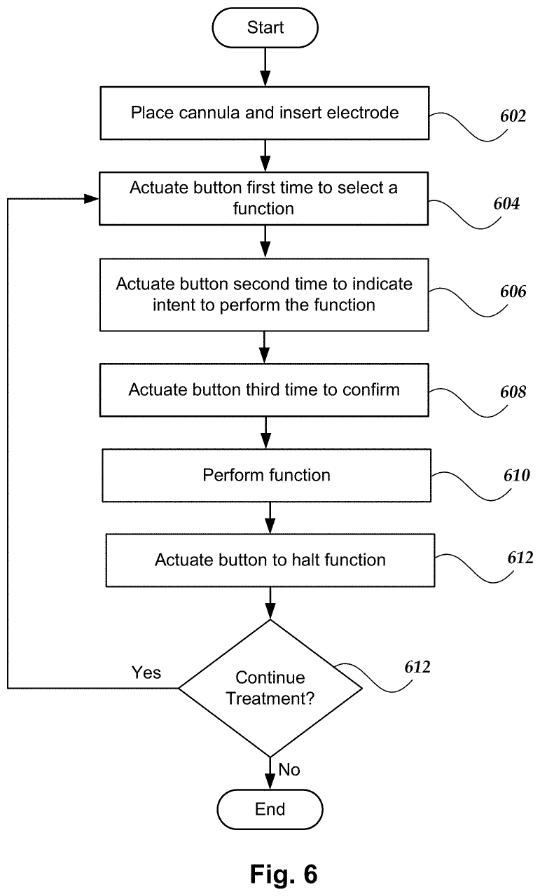

FIG. 6 illustrates one method of using the in-line controller 240. In step 602, the cannula 106 is placed in the patient and the RF electrode 104 is inserted in the cannula. The RF generator 102 is also turned on.

In step 604, a button 242 of the in-line controller 240 is actuated to select a function. In at least some embodiments, the function is selected by selecting a button 242 from a set of multiple buttons. In other embodiments, the function can be selected using the button 242 to select a function from a menu on the RF generator 102.

In step 606, the button 242 of the in-line controller 240 is actuated again to indicate the intent to perform the function. In other embodiments, this step may be deleted and the first actuation of the button 242 is sufficient to indicate intent to perform the function. In at least some embodiments, after the intent is indicated, the RF generator 102 displays a request for confirmation of the function.

In step 608, the button 242 of the in-line controller 240 is actuated once more to confirm that the function is to be performed. In step 610, the RF generator 102 performs the function.

In step 612, the button 242 of the in-line controller 240 is actuated to halt the function. In step 614, the system or user determines if treatment is to continue. If so, the system returns to step 604 to await actuation of a button. If not, then the method ends.

As an example, the clinician can place the cannula and electrode. The clinician can press a button three times to select, indicate, and confirm the motor stimulation function. After sufficient motor stimulation, the clinician can press the button again to halt that function. Then, the clinician can press a button three times to select, indicate, and confirm the thermal ablation function. After sufficient motor stimulation, the clinician can press the button again to halt that function. In at least some embodiments, the thermal ablation function may initiate an additional warning screen on the RF generator to indicate the generator has autoramp on. The clinician may be required to press the button an extra time to dismiss this warning screen and begin the thermal ablation.

In at least some embodiments, the RF generator 102 may be configured so that if multiple buttons 242 are depressed at the same time, the current function ends as a safety feature and a warning screen may be presented on the RF generator which may be dismissed by depressing any individual button.

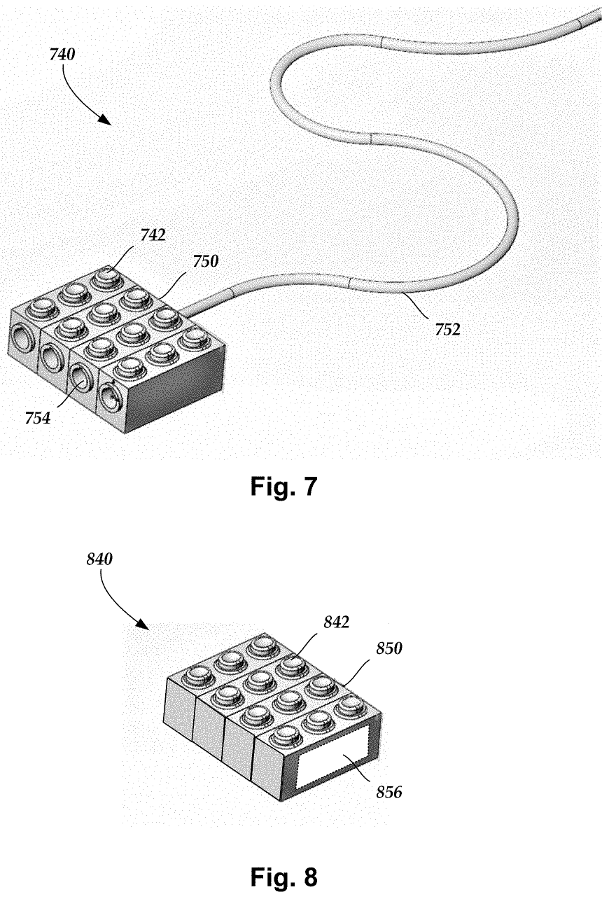

FIG. 7 illustrates another in-line controller 740 having a controller body 750 with multiple buttons 742 and a cable 752 that attaches to the RF generator 102. The cable 752 can be a master extension cable. In at least some embodiments, the remote controller 740 has multiple ports 754 for the RF electrodes 104. Each port 754 is associated with one or more of the buttons 742 that can be used in the same manner as the buttons 242 of the in-line controller 240. The remote controller 750 can be positioned closer to the cannula 106 so that it is easier to identify which RF electrode 104 goes to which port 754. In at least some embodiments, the buttons 742 of the remote controller 740 can have different colors or different shapes to differentiate the buttons' function.

The in-line controller 240 and in-line controller 740 act as a remote controller for the RF generator 102. Another embodiment of a remote controller 840, which is similar to in-line controller 740, includes the controller body 850 and buttons 842 as illustrated in FIG. 8. However, the remote controller 840 utilizes a wireless communication module 856 (for example, Bluetooth™ communication circuitry) for communication to the RF generator 102. Such a remote controller 740 may not include ports, but rather each button 842 or group of buttons would be associated with a particular port 122 of the RF generator 102. As an alternative to the wireless communication module 856, the remote controller 840 can be attached to the RF generator 102 by a cable.

In at least some embodiments, the remote controller 740, 840 can include indicator lights or a small visual screen to provide visual feedback on the performance of the RF generator 102.

The above specification provides a description of the structure, manufacture, and use of the invention. Since many embodiments of the invention can be made without departing from the spirit and scope of the invention, the invention also resides in the claims hereinafter appended.

What is claimed as new and desired to be protected by Letters Patent of the United States is:

1. A RF ablation system, comprising:
a RF electrode coupleable to a RF generator for delivering RF energy from the RF generator to patient tissue for ablation, the RF electrode comprising an electrode hub, an electrode shaft extending from the electrode hub, and a cable extending from the electrode hub; and
an in-line controller coupleable to the RF generator for controlling the delivering of the RF energy from the RF generator along the RF electrode, wherein the in-line controller is configured for placement nearer the electrode hub than the RF generator and comprises at least one actuator for controlling the delivering of the RF energy.

2. The RF ablation system of claim 1, wherein the in-line controller is disposed along the cable of the RF electrode.

3. The RF ablation system of claim 2, wherein the in-line controller is disposed in a range of 4 to 160 cm along the cable from the electrode hub.

4. The RF ablation system of claim 1, further comprising an extension cable configured to couple the cable of the RF electrode to the RF generator.

5. The RF ablation system of claim 4, wherein the in-line controller is disposed along the extension cable.

6. The RF ablation system of claim 1, wherein the in-line controller is disposed on the electrode hub.

7. The RF ablation system of claim 1, wherein the at least one actuator comprises a plurality of user-operable buttons.

8. The RF ablation system of claim 7, wherein the user-operable buttons are color-coded.

9. The RF ablation system of claim 7, wherein the user-operable buttons are squeeze sections of the cable of the RF electrode.

10. The RF ablation system of claim 1, wherein the in-line controller comprises a controller body, wherein the at least one actuator comprises a plurality of user-operable buttons disposed along the controller body.

11. The RF ablation system of claim 10, wherein the RF electrode further comprises a connector disposed at an end of the cable and the controller body comprises a plurality of ports configured to receive the connector from the RF electrode.

12. The RF ablation system of claim 11, wherein multiple ones of the user-operable buttons are individually associated with each of the ports.

13. The RF ablation system of claim 1, further comprising the RF generator coupleable to the RF electrode and the in-line controller.

14. A RF electrode coupleable to a RF generator for delivering RF energy from the RF generator to patient tissue for ablation, the RF electrode comprising
an electrode hub;
an electrode shaft extending from the electrode hub;
a cable extending from the electrode hub; and
an in-line controller disposed along the cable and coupleable to the RF generator for controlling the delivering of the RF energy from the RF generator along the RF electrode.

15. The RF ablation system of claim 14, wherein the in-line controller is disposed in a range of 4 to 10 cm along the cable from the electrode hub.

16. The RF ablation system of claim 14, wherein the in-line controller is disposed on the electrode hub.

17. The RF ablation system of claim 14, wherein the at least one actuator comprises a plurality of user-operable buttons.

18. The RF ablation system of claim 17, wherein the user-operable buttons are color-coded.

19. The RF ablation system of claim 17, wherein the user-operable buttons are squeeze sections of the cable of the RF electrode.

20. A remote controller for a RF generator for providing RF ablation to a patient, the remote controller comprises:
a controller body;
a plurality of user-operable buttons disposed along the controller body, wherein each of the user-operable buttons is associated with one of a plurality of RF channels of the RF generator; and
a wireless communication module configured for wireless communication with the RF generator.
